(19) United States
(12) Patent Application Publication
(10) Pub. No.: US 2016/0077114 A1
(43) Pub. Date: Mar. 17, 2016

(54) METHODS FOR PREDICTING AMOUNTS OF OSTEOACTIVITY MARKERS

(71) Applicant: Community Blood Center, Dayton, OH (US)

(72) Inventors: Mary E. Blackmore, Dayton, OH (US); Felicia C.T. Gooden, Beavercreek, OH (US); Shawn A. Hunter, Springboro, OH (US)

(73) Assignee: Community Blood Center, Dayton, OH (US)

(21) Appl. No.: 14/855,694

(22) Filed: Sep. 16, 2015

Related U.S. Application Data
(60) Provisional application No. 62/051,568, filed on Sep. 17, 2014.

(51) Int. Cl. G01N 33/74; G01N 33/573; G01N 33/68

(52) U.S. Cl. CPC G01N 33/74; G01N 33/6803; G01N 33/573; G01N 2333/4703; G01N 2333/51; G01N 2333/916

(57) ABSTRACT

Various embodiments describe methods of predicting an amount of a target osteoactivity marker. The methods include measuring an initial concentration of the target osteoactivity marker at a time t₀; measuring an initial concentration of at least one additional osteoactivity marker at the time t₀; and calculating a second concentration of the target osteoactivity marker at a time t₁ based on the initial concentration of the target osteoactivity marker at the time t₀ and the initial concentration of the at least one additional osteoactivity marker at the time t₀.

### CROSS-REFERENCE TO RELATED APPLICATIONS

This patent application claims the benefit of Provisional Patent Application Ser. No. 62/051,568 filed on Sep. 17, 2014, which is hereby incorporated by reference in its entirety.

### TECHNICAL FIELD

The present specification generally relates to osteoactivity and, more specifically, to methods for predicting amounts of osteoactivity markers.

### BACKGROUND

Because most in vivo diagnostic tests for product characterization and determination of osteoinductivity are time-consuming and costly, alternative in vitro testing is typically conducted using an assay of alkaline phosphatase (ALP), an enzyme that dephosphorylates other molecules and is tissue non-specific, or via direct measurement of BMPs. However, such assays may only provide limited information regarding the specific target.

Accordingly, a need exists for a method for predicting amounts of multiple osteoactivity markers from limited information while avoiding the non-specificity, time, and costs associated with conventional assays.

### SUMMARY

According to one embodiment, a method of predicting an amount of a target osteoactivity marker includes measuring an initial concentration of the target osteoactivity marker at a time t₀; measuring an initial concentration of at least one additional osteoactivity marker at the time t₀; and calculating a second concentration of the target osteoactivity marker at a time t₁ based on the initial concentration of the target osteoactivity marker at the time t₀ and the initial concentration of the at least one additional osteoactivity marker at the time t₀.

According to another embodiment, a method of predicting an amount of a target osteoactivity marker includes receiving a sample comprising biological material; measuring an initial concentration of the target osteoactivity marker in the sample at a time t₀; measuring an initial concentration of at least one additional osteoactivity marker in the sample at the time t₀; calculating a second concentration of the target osteoactivity marker at a time t₁ based on the initial concentration of the target osteoactivity marker at the time t₀ and the initial concentration of the at least one additional osteoactivity marker at the time t₀; and reporting an output comprising information regarding osteoactivity of the sample.

Additional features and advantages will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 schematically depicts the osteoblastic pathway including one or more osteoactivity markers in accordance with one or more embodiments described herein;

FIG. 2 illustrates a plot showing theoretical and experimental rates of change of BMP2 in accordance with one or more embodiments described herein;

FIG. 3 illustrates a plot showing theoretical and experimental rates of change of BMP7 in accordance with one or more embodiments described herein;

FIG. 4 illustrates a plot showing theoretical and experimental rates of change of BMP8a in accordance with one or more embodiments described herein;

FIG. 5 illustrates a plot showing theoretical and experimental rates of change of pSMAD in accordance with one or more embodiments described herein;

FIG. 6 illustrates a plot showing theoretical and experimental rates of change of DLX5 in accordance with one or more embodiments described herein;

FIG. 7 illustrates a plot showing theoretical and experimental rates of change of RUNX2 in accordance with one or more embodiments described herein;

FIG. 8 illustrates a plot showing theoretical and experimental rates of change of SP7 in accordance with one or more embodiments described herein; and

FIG. 9 illustrates a plot showing theoretical and experimental rates of change of ALP in accordance with one or more embodiments described herein.

### DETAILED DESCRIPTION

Reference will now be made in detail to various embodiments of methods for predicting amounts of osteoactivity markers. In general, various embodiments enable concentrations of one or more osteoactivity markers to be predicted based on an initial, measured concentration of the osteoactivity markers. Accordingly, concentrations of osteoactivity markers may be acquired at a single point in time and a concentration of a target osteoactivity marker may be reliably predicted at other points in time based on the initial concentrations. Such methods may enable the assay to be performed one time, which in turn may save time and money, and require smaller sample sizes.

In various embodiments, methods for predicting an amount of a target osteoactivity marker include measuring an initial concentration of the target osteoactivity marker at a time t₀, measuring an initial concentration of at least one additional osteoactivity marker at the time t₀, and calculating a second concentration of the target osteoactivity marker at a time t₁ based on the initial concentration of the target osteoactivity marker and the initial concentration of the at least one additional osteoactivity marker at the time t₀.

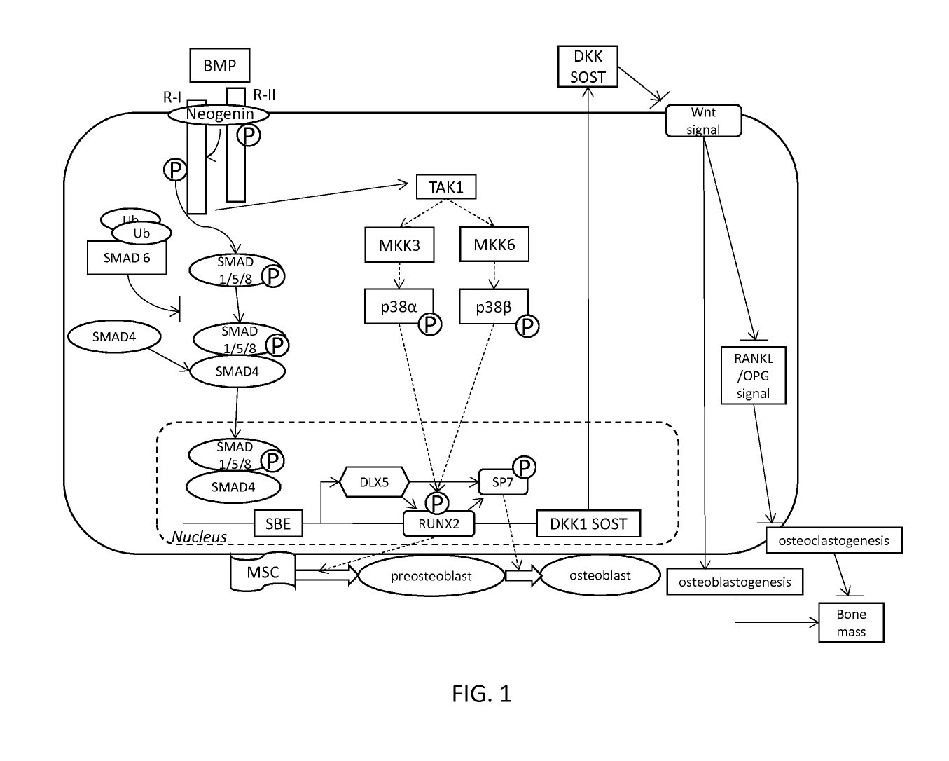

Osteoactivity markers include various components of the osteoblastic pathway, such as enzymes and proteins. By way of example and not limitation, osteoactivity markers may include phosphorylated small body size mothers against decapentapletic 1/5 (pSMAD 1/5), runt-related transcription factor 2 (RUNX2), distal-less homeobox 5 (DLX5), osterix (SP7 or Osx), alkaline phosphatase (ALP), bone morphogenetic protein 2 (BMP2), bone morphogenetic protein 7 (BMP7), and bone morphogenetic protein 8a (BMP8a). FIG. 1 graphically depicts various components in the osteoblastic pathway.

Without being bound by theory, pSMAD 1/5 proteins are receptor-regulated intracellular proteins that transduce extracellular signals from BMPs 2, 7, and 8a to activate downstream gene transcription. Accordingly, pSMAD 1/5 protein levels increase over time as the result of BMP phosphorylation of small body size mothers against decapentapletic 1/5 (SMAD 1/5ᵤₙ) and decrease as a result of cytoplasmic export, its role in the production of downstream proteins, and degradation.

RUNX2 is a protein encoded by the RUNX2 gene and, without being bound by theory, is a key transcription factor in osteoblast differentiation. RUNX2 is believed to contribute to SP7 and ALP production. DLX5 is a protein encoded by the DLX5 gene and, without being bound by theory, is an early BMP-responsive transcriptional activator essential to osteoblast differentiation due to its activities in up-regulating the production of downstream proteins, including RUNX2 and SP7. SP7 is a zinc finger transcription factor that is believed to be responsible for regulating bone cell differentiation. SP7 may contribute to ALP production.

BMPs 2, 7, and 8a are members of the transforming growth factor-β (TGF-β) family of proteins. Without being bound by theory, the TGF-β family of proteins plays a key role in the formation of bone and cartilage. BMPs 2 and 7 are considered to be osteoinductive due to their ability to promote osteoblastic differentiation in numerous cell types. BMP2 is produced directly via transcription and translation of the BMP2 gene. BMP7, which may also play a key role in bone homeostasis maintenance by induction of the phosphorylation of SMADs 1 and 5, is produced directly via transcription and translation of the BMP7 gene. Similar to BMPs 2 and 7, BMP8a is a disulfide-linked homodimeric protein produced directly via transcription and translation of its gene. Like the other BMPs described herein, BMP8a may be involved in bone and cartilage development and in bone homeostasis.

In various embodiments, the osteoactivity markers are connected by Michaelis-Menten kinetic equations used to describe the osteoblastic pathway. Accordingly, the concentrations of various osteoactivity markers may be computed at various points in time depending on initial measured concentrations.

Concentrations of osteoactivity markers may be acquired according to various methods, including measurement based on osteoactivity markers extracted from a sample or prediction. The sample may be, for example, biological material that may or may not contain cell suspensions, tissue extracts, blood, urine, media in which cells were grown, and/or cellular waste. Depending on the particular embodiment, the sample may be prepared according to techniques known in the art for protein extraction and quantification.

In various embodiments, an initial concentration of an osteoactivity marker of interest and one or more additional osteoactivity markers may be measured using one or more assays. In some embodiments, two or more additional osteoactivity markers or three or more additional osteoactivity markers may be measured in addition to the osteoactivity marker of interest. For example, manual or automated Western blots may be employed to measure an initial concentration of a target osteoactivity marker of interest and an initial concentration of one or more additional osteoactivity markers. In further embodiments, an initial concentration of one or more osteoactivity markers may be acquired by predicting the concentration of the osteoactivity marker according to various embodiments provided herein. In various embodiments, the initial concentrations of the osteoactivity markers are taken at a time t₀.

Based on the initial concentrations of the osteoactivity markers, concentrations at other points in time may be predicted. In particular, in various embodiments, the initial concentrations may be input into a computing device that may calculate a concentration at another time, t₁. More specifically, data handling software may be employed to execute computer-readable instructions that cause a processor of the computing device to calculate a concentration at a time t₁ based on initial concentrations of the osteoactivity marker of interest and of one or more additional osteoactivity marker.

In various embodiments, the osteoactivity marker of interest is pSMAD 1/5. In such embodiments, initial concentrations of pSMAD 1/5, BMP2, BMP7, BMP8a, and SMAD 1/5ᵤₙ at time t₀ are acquired for the sample. Then, the rate of change of the concentration of pSMAD 1/5 is calculated according to the following equation:

$$\frac{d [ pSMAD 1 / 5 ]}{d t} = ( \frac{{v}_{1} [ BMP 2 ]}{{K}_{1} + [ BMP 2 ]} + \frac{{v}_{2} [ BMP 7 ]}{{K}_{2} + [ BMP 7 ]} + \frac{{v}_{3} [ BMP 8 a ]}{{K}_{3} + [ BMP 8 a ]} ) [ SMAD 1 / {5}_{un} ] - {d}_{1} [ pSMAD 1 / 5 ]$$

where v₁ is the maximum velocity of pSMAD 1/5 as the product of BMP2, v₂ is the maximum velocity of pSMAD 1/5 as the product of BMP7, v₃ is the maximum velocity of pSMAD 1/5 as the product of BMP8a, K₁ is the Michaelis constant of pSMAD 1/5 associated with BMP2, K₂ is the Michaelis constant of pSMAD 1/5 associated with BMP7, K₃ is the Michaelis constant of pSMAD 1/5 associated with BMP8a, d₁ is the depletion rate of pSMAD 1/5, [BMP2] is the concentration of BMP2 at a time t₀, [BMP7] is the concentration of BMP7 at the time t₀, [BMP8a] is the concentration of BMP8a at the time t₀, [SMAD1/5ᵤₙ] is the concentration of SMAD 1/5ᵤₙ at the time t₀, and [pSMAD] is the concentration of pSMAD at the time t₀.

Each of the maximum velocities is measured in concentration over time, and each of the Michaelis constants is in concentration. The depletion rate is measured in (1/time). In various embodiments, v₁ is between about 5.0 and about 6.0 or between about 5.05 and about 5.50, v₂ is between about 0.7 and about 2.0 or between about 1.0 and about 1.5, and v₃ is between about 0.1 and about 2.0 or between about 0.8 and about 1.2. K₁ may be from about 0.001 to about 1.0 or from about 0.01 to about 0.10, K₂ may be from about 0.001 to about 1.0 or from about 0.01 to about 0.10, and K₃ may be from about 0.0001 to about 1.0 or from about 0.001 to about 0.002. Additionally, d₁ may be from about 0.001 to about 1.0 or from about 0.01 to about 0.02.

In various embodiments, each of v₁, v₂, v₃, K₁, K₂, K₃, and d₁ may be constants that are determined from various experiments and optimized. For example, in some embodiments, equations and raw data may be incorporated into an optimization algorithm, such as the Generalized Reduced Gradient (GRG) nonlinear algorithms, to identify optimal values for the constants. In some embodiments, the values selected for each of the constants may permit the models to maintain accuracy across various cell types.

In embodiments in which the osteoactivity marker of interest is RUNX2, initial concentrations of pSMAD 1/5, DLX5, and RUNX2 at time t₀ are acquired for the sample. Then, the rate of change of the concentration of RUNX2 is calculated according to the following equation:

$$\frac{d [ RUNX 2 ]}{d t} = ( \frac{{v}_{4} [ pSMAD 1 / 5 ]}{{K}_{4} + [ pSMAD 1 / 5 ]} + \frac{{v}_{5} [ DLX 5 ]}{{K}_{5} + [ DLX 5 ]} ) - {d}_{2} [ RUNX 2 ]$$

where v₄ is the maximum velocity of RUNX2 as the product of pSMAD 1/5, v₅ is the maximum velocity of RUNX2 as the product of DLX5, K₄ is the Michaelis constant of RUNX2 associated with pSMAD 1/5, K₅ is the Michaelis constant of RUNX2 associated with DLX5, d₂ is the depletion rate of RUNX2, [RUNX2] is the concentration of RUNX2 at a time t₀, [DLX5] is the concentration of DLX5 at the time t₀, and [pSMAD] is the concentration of pSMAD at the time t₀.

Each of the maximum velocities is measured in concentration over time, and each of the Michaelis constants is in concentration. The depletion rate is measured in (1/time). In various embodiments, v₄ is between about 3.0 and about 10.0 or between about 4.0 and about 6.0 and v₅ is between about 8.0 and about 12.0 or between about 8.5 and about 9.5. K₄ may be from about 4000 to about 6000 or from about 5000 to about 5700 and K₅ may be from about 0.001 to about 1.0 or from about 0.1 to about 0.5. Additionally, d₂ may be from about 0.0001 to about 1.0 or from about 0.001 to about 0.005.

As above, each of v₄, v₅, K₄, K₅, and d₂ may be constants that are determined from various experiments and optimized. For example, in some embodiments, equations and raw data may be incorporated into an optimization algorithm, such as the Generalized Reduced Gradient (GRG) nonlinear algorithms, to identify optimal values for the constants.

In embodiments in which the osteoactivity marker of interest is DLX5, initial concentrations of pSMAD 1/5 and DLX5 at time t₀ are acquired for the sample. Then, the rate of change of the concentration of DLX5 is calculated according to the following equation:

$$\frac{d [ DLX 5 ]}{d t} = ( \frac{{v}_{6} [ pSMAD 1 / 5 ]}{{K}_{6} + ( pSMAD 1 / 5 ]} ) - {d}_{3} [ DLX 5 ]$$

where v₅ is the maximum velocity of DLX5 as the product of pSMAD 1/5, K₅ is the Michaelis constant of DLX5 associated with pSMAD 1/5, d₃ is the depletion rate of DLX5, [DLX5] is the concentration of DLX5 at the time t₀, and [pSMAD] is the concentration of pSMAD at the time t₀.

Each of the maximum velocities is measured in concentration over time, and each of the Michaelis constants is in concentration. The depletion rate is measured in (1/time). In various embodiments, v₆ is between about 0.1 and about 170.0 or between about 1.0 and about 100.0 or between about 10 and about 30, and K₆ may be from about 0.001 to about 61.0 or from about 1 to about 40 or from about 10 to about 30. Additionally, d₃ may be from about 0.0001 to about 1.0 or from about 0.001 to about 0.005.

As above, each of V₆, K₆, and d₃ may be constants that are determined from various experiments and optimized. For example, in some embodiments, equations and raw data may be incorporated into an optimization algorithm, such as the Generalized Reduced Gradient (GRG) nonlinear algorithms, to identify optimal values for the constants.

In some embodiments, the osteoactivity marker of interest is SP7. In such embodiments, initial concentrations of SP7, pSMAD 1/5, DLX5, and RUNX2 at time t₀ are acquired for the sample. Then, the rate of change of the concentration of SP7 is calculated according to the following equation:

$$\frac{d [ SP 7 ]}{d t} = ( \frac{{v}_{7} [ pSMAD 1 / 5 ]}{{K}_{7} + [ pSMAD 1 / 5 ]} + \frac{{v}_{8} [ DLX 5 ]}{{K}_{8} + [ DLX 5 ]} + \frac{{v}_{9} [ RUNX 2 ]}{{K}_{9} + [ RUNX 2 ]} ) - {d}_{4} [ SP 7 ]$$

where v₇ is the maximum velocity of SP7 as the product of pSMAD 1/5, v₈ is the maximum velocity of SP7 as the product of DLX5, v₉ is the maximum velocity of SP7 as the product of RUNX2, K₇ is the Michaelis constant of SP7 associated with pSMAD 1/5, K₈ is the Michaelis constant of SP7 associated with DLX5, K₉ is the Michaelis constant of SP7 associated with RUNX2, d₃ is the depletion rate of SP7, [SP7] is the concentration of SP7 at a time t₀, [RUNX2] is the concentration of RUNX2 at the time t₀, [DLX5] is the concentration of DLX5 at the time t₀, and [pSMAD] is the concentration of pSMAD at the time t₀.

Each of the maximum velocities is measured in concentration over time, and each of the Michaelis constants is in concentration. The depletion rate is measured in (1/time). In various embodiments, v₇ is between about 0 and about 276.0 or between about 75 and about 150, v₈ is between about 0.001 and about 1.0 or between about 0.01 and about 0.10, and v₉ is between about 0.001 and about 37.0 or between about 5 and about 25. K₇ may be from about 0.001 to about 1.0 or from about 0.10 to about 0.50, K₈ may be from about 0.01 to about 40.0 or from about 1.0 to about 38, and K₉ may be from about 0.1 to about 3.0 or from about 1.0 to about 2.8. Additionally, d₄ may be from about 0 to about 1.0 or from about 0.001 to about 0.01.

As above, each of v₇, v₈, V₉, K₇, K₈, K₉, and d₄ may be constants that are determined from various experiments and optimized. For example, in some embodiments, equations and raw data may be incorporated into an optimization algorithm, such as the Generalized Reduced Gradient (GRG) nonlinear algorithms, to identify optimal values for the constants.

In some embodiments, the osteoactivity marker of interest is BMP2. In such embodiments, initial concentrations of BMP2, SP7, DLX5, and RUNX2 at time t₀ are acquired for the sample. Then, the rate of change of the concentration of BMP2 is calculated according to the following equation:

$$\frac{d [ BMP 2 ]}{d t} = {r}_{10} ( \frac{{v}_{10} [ RUNX 2 ]}{{K}_{10} + [ RUNX 2 ]} ) + {r}_{11} ( \frac{{v}_{11} [ DLX 5 ]}{{K}_{11} + [ DLX 5 ]} ) + {r}_{12} ( \frac{{v}_{12} [ SP 7 ]}{{K}_{12} + [ SP 7 ]} ) - {d}_{5} [ BMP 2 ]$$

where r₁₀ is a contribution coefficient of RUNX2 to the production of BMP2, r₁₁ is a contribution coefficient of DLX5 to the production of BMP2, r₁₂ is a contribution coefficient of SP7 to the production of BMP2, v₁₀ is the maximum velocity of BMP2 as the product of RUNX2, v₁₁ is the maximum velocity of BMP2 as the product of DLX5, v₁₂ is the maximum velocity of BMP2 as the product of SP7, K₁₀ is the Michaelis constant of BMP2 associated with RUNX2, K₁₁ is the Michaelis constant of BMP2 associated with DLX5, K₁₂ is the Michaelis constant of BMP2 associated with SP7, d₅ is the depletion rate of BMP2, [SP7] is the concentration of SP7 at a time t₀, [RUNX2] is the concentration of RUNX2 at the time t₀, [DLX5] is the concentration of DLX5 at the time t₀, and [BMP2] is the concentration of BMP2 at the time t₀.

Each of the maximum velocities is measured in concentration over time, and each of the Michaelis constants is in concentration. The depletion rate is measured in (1/time). In various embodiments, v₁₀ is between about 0 and about 1.0 or between about 2.0×10⁻⁵ and about 1×10⁻⁴, v₁₁ is between about 0 and about 1.0 or between about 0.01 and about 0.10, and v₁₂ is between about 20.0 and about 53.0 or between about 30.0 and about 40.0. K₁₀ may be from about 0.001 to about 1.0 or from about 0.01 to about 0.04, K₁₁ may be from about 0 to about 1.0 or from about 5×10⁻⁵ to about 1×10⁻⁴, and K₁₂ may be from about 0 to about 1.0 or from about 0.01 to about 0.03. Additionally, r₁₀ may be from about 0.001 to about 1.0 or from about 0.003 to about 0.01, r₁₁ may be from about 0.0001 to about 1 or from about 0.001 to about 0.010, r₁₂ may be from about 0.001 to about 1.0 or from about 0.01 to about 0.5, and d₅ may be from about 0.0001 to about 1.0 or from about 0.001 to about 0.01.

As above, each of v₁₀, v₁₁, v₁₂, K₁₀, K₁₁, K₁₂, r₁₀, r₁₁, r₁₂, and d₅ may be constants that are determined from various experiments and optimized. For example, in some embodiments, equations and raw data may be incorporated into an optimization algorithm, such as the Generalized Reduced Gradient (GRG) nonlinear algorithms, to identify optimal values for the constants.

In still other embodiments, the osteoactivity marker of interest is BMP7. In such embodiments, initial concentrations of BMP7, SP7, DLX5, and RUNX2 at time t₀ are acquired for the sample. Then, the rate of change of the concentration of BMP7 is calculated according to the following equation:

$$\frac{d [ BMP 7 ]}{d t} = {r}_{13} ( \frac{{v}_{13} [ RUNX 2 ]}{{K}_{13} + [ RUNX 2 ]} ) + {r}_{14} ( \frac{{v}_{14} [ DLX 5 ]}{{K}_{14} + [ DLX 5 ]} ) + {r}_{15} ( \frac{{v}_{15} [ SP 7 ]}{{K}_{15} + [ SP 7 ]} ) - {d}_{6} [ BMP 7 ]$$

where r₁₃ is a contribution coefficient of RUNX2 to the production of BMP7, r₁₄ is a contribution coefficient of DLX5 to the production of BMP7, r₁₅ is a contribution coefficient of SP7 to the production of BMP7, v₁₃ is the maximum velocity of BMP7 as the product of RUNX2, v₁₄ is the maximum velocity of BMP7 as the product of DLX5, v₁₅ is the maximum velocity of BMP7 as the product of SP7, K₁₃ is the Michaelis constant of BMP7 associated with RUNX2, K₁₄ is the Michaelis constant of BMP7 associated with DLX5, K₁₅ is the Michaelis constant of BMP7 associated with SP7, d₆ is the depletion rate of BMP7, [SP7] is the concentration of SP7 at a time t₀, [RUNX2] is the concentration of RUNX2 at the time t₀, [DLX5] is the concentration of DLX5 at the time t₀, and [BMP7] is the concentration of BMP7 at the time t₀.

Each of the maximum velocities is measured in concentration over time, and each of the Michaelis constants is in concentration. The depletion rate is measured in (1/time). In various embodiments, v₁₃ is between about 0 and about 1.0 or between about 2.0×10⁻⁵ and about 1×10⁻⁴, v₁₄ is between about 0.1 and about 6.0 or between about 3.0 and about 5.0, and v₁₅ is between about 80.0 and about 90.0 or between about 83.0 and about 87.0. K₁₃ may be from about 0.01 to about 1.0 or from about 0.1 to about 0.5, K₁₄ may be from about 0 to about 1.0 or from about 1.5×10⁻⁵ to about 1×10⁻⁴, and K₁₅ may be from about 0 to about 1.0 or from about 5×10⁻⁵ to about 5×10⁻⁴. Additionally, r₁₃ may be from about 0.0001 to about 1.0 or from about 0.003 to about 0.01, r₁₄ may be from about 0.001 to about 1 or from about 0.01 to about 0.1, r₁₅ may be from about 0.001 to about 1.0 or from about 0.01 to about 0.5, and d₆ may be from about 0.001 to about 1.0 or from about 0.01 to about 0.5.

As above, each of v₁₃, v₁₄, V₁₅, K₁₃, K₁₄, K₁₅, r₁₃, r₁₄, r₁₅, and d₆ may be constants that are determined from various experiments and optimized. For example, in some embodiments, equations and raw data may be incorporated into an optimization algorithm, such as the Generalized Reduced Gradient (GRG) nonlinear algorithms, to identify optimal values for the constants.

In still other embodiments, the osteoactivity marker of interest is BMP8a. In such embodiments, initial concentrations of BMP7, SP7, DLX5, and RUNX2 at time t₀ are acquired for the sample. Then, the rate of change of the concentration of BMP7 is calculated according to the following equation:

$$\frac{d [ BMP 8 a ]}{d t} = {r}_{16} ( \frac{{v}_{16} [ RUNX 2 ]}{{K}_{16} + [ RUNX 2 ]} ) + {r}_{17} ( \frac{{v}_{17} [ DLX 5 ]}{{K}_{17} + [ DLX 5 ]} ) + {r}_{18} ( \frac{{v}_{18} [ SP 7 ]}{{K}_{18} + [ SP 7 ]} ) - {d}_{7} [ BMP 8 a ]$$

where r₁₆ is a contribution coefficient of RUNX2 to the production of BMP8a, r₁₇ is a contribution coefficient of DLX5 to the production of BMP8a, r₁₈ is a contribution coefficient of SP7 to the production of BMP8a, v₁₆ is the maximum velocity of BMP8a as the product of RUNX2, v₁₇ is the maximum velocity of BMP8a as the product of DLX5, v₁₈ is the maximum velocity of BMP8a as the product of SP7, K₁₆ is the Michaelis constant of BMP8a associated with RUNX2, K₁₇ is the Michaelis constant of BMP8a associated with DLX5, K₁₈ is the Michaelis constant of BMP8a associated with SP7, d₇ is the depletion rate of BMP8a, [SP7] is the concentration of SP7 at a time t₀, [RUNX2] is the concentration of RUNX2 at the time t₀, [DLX5] is the concentration of DLX5 at the time t₀, and [BMP8a] is the concentration of BMP8a at the time t₀.

Each of the maximum velocities is measured in concentration over time, and each of the Michaelis constants is in concentration. The depletion rate is measured in (1/time). In various embodiments, v₁₆ is between about 14.0 and about 60.0, between about 25 and about 55, or between about 40 and about 50, v₁₇ is between about 0 and about 5.0 or between about 0 and about 1.0, and v₁₈ is between about 0 and about 1.0 or between about 1×10⁻¹² and about 1×10⁻⁵. K₁₆ may be from about 0 to about 1.0 or from about 0.1 to about 0.5, K₁₇ may be from about 0 to about 1.0 or from about 1.5×10⁻⁵ to about 1×10⁻⁴, and K₁₈ may be from about 0 to about 1.0 or from about 1×10⁻⁴ to about 0.001. Additionally, r₁₆ may be from about 0.01 to about 2.0 or from about 0.5 to about 2.0, r₁₇ may be from about 0 to about 5.0 or from about 0 to about 0.1, r₁₈ may be from about 0.0001 to about 1.0 or from about 0.0001 to about 0.01, and d₇ may be from about 0.001 to about 1.0 or from about 0.01 to about 0.5.

As above, each of v₁₆, v₁₇, V₁₈, K₁₆, K₁₇, K₁₈, r₁₆, r₁₇, r₁₈, and d₇ may be constants that are determined from various experiments and optimized. For example, in some embodiments, equations and raw data may be incorporated into an optimization algorithm, such as the Generalized Reduced Gradient (GRG) nonlinear algorithms, to identify optimal values for the constants.

In various embodiments, the coefficients r₁₀, r₁₁, r₁₂, r₁₃, r₁₄, r₁₅, r₁₆, r₁₇, and r₁₈ are further constrained according to the following equations:

r₁₀+r₁₃+r₁₆≦1.0;

r₁₁+r₁₄+r₁₇≦1.0; and

r₁₂+r₁₅+r₁₈≦1.0.

Without being bound by theory, r₁₀, r₁₃, and r₁₆ are associated with the contribution of RUNX2 to the production of BMPs 2, 7, and 8a through feedback, so these values cannot sum to a number greater than 1.0 because there cannot be greater than 100% of the RUNX2 that is depleted from the system consumed in the production of BMPs. Similarly, without being bound by theory, r₁₁, r₁₄, and r₁₇ are associated with the contribution of DLX5 to the production of BMPs 2, 7, and 8a through feedback, so these values cannot sum to a number greater than 1.0 because there cannot be greater than 100% of the DLX5 that is depleted from the system consumed in the production of BMPs. Moreover, without being bound by theory, r₁₂, r₁₅, and r₁₈ are associated with the contribution of SP7 to the production of BMPs 2, 7, and 8a through feedback, so these values cannot sum to a number greater than 1.0 because there cannot be greater than 100% of the SP7 that is depleted from the system consumed in the production of BMPs.

In embodiments in which the osteoactivity marker of interest is ALP, initial concentrations of ALP, SP7, and RUNX2 at time t₀ are acquired for the sample. Then, the rate of change of the concentration of ALP is calculated according to the following equation:

$$\frac{d [ ALP ]}{d t} = ( \frac{{v}_{20} [ RUNX 2 ]}{{K}_{20} + [ RUNX 2 ]} + \frac{{v}_{21} [ SP 7 ]}{{K}_{21} + [ SP 7 ]} ) - {d}_{8} [ ALP ]$$

where v₂₀ is the maximum velocity of ALP as the product of RUNX2, v₂₁ is the maximum velocity of ALP as the product of SP7, K₂₀ is the Michaelis constant of ALP associated with RUNX2, K₂₁ is the Michaelis constant of ALP associated with SP7, d₈ is the depletion rate of ALP, [RUNX2] is the concentration of RUNX2 at a time t₀, [SP7] is the concentration of SP7 at the time t₀, and [ALP] is the concentration of ALP at the time t₀.

Each of the maximum velocities is measured in concentration over time, and each of the Michaelis constants is in concentration. The depletion rate is measured in (1/time). In various embodiments, v₂₀ is between about 0.001 and about 10,165.0 or between about 0.1 and about 2.0 and v₂₁ is between about 0.001 and about 1.0 or between about 0.1 and about 0.5. K₂₀ may be from about 0.001 to about 1.0 or from about 0.005 to about 0.1 and K₂₁ may be from about 0 to about 1.0 or from about 1×10⁻¹° to about 1×10⁻⁴. Additionally, d₈ may be from about 0.0001 to about 1.0 or from about 0.001 to about 0.005.

As above, each of v₄, v₅, K₄, K₅, and d₂ may be constants that are determined from various experiments and optimized. For example, in some embodiments, equations and raw data may be incorporated into an optimization algorithm, such as the Generalized Reduced Gradient (GRG) nonlinear algorithms, to identify optimal values for the constants.

For each of the above-described embodiments, once the d/dt for the particular osteoactivity marker has been calculated, the concentration of the osteoactivity marker at any other point in time can be calculated. Accordingly, a second concentration of the osteoactivity marker at a time t₁ can be calculated based on the initial concentration of the osteoactivity marker and the initial concentration(s) of at least one additional osteoactivity marker.

In some embodiments, in addition to calculating a second concentration of the osteoactivity marker, a second concentration of at least one additional osteoactivity marker may be calculated at a time t₁. For example, the concentration of BMP2, BMP7, and BMP8a may be calculated first, which can then be used to calculate a concentration of pSMAD 1/5, which can be used to calculate a concentration of DLX5, which can be used to calculate a concentration of RUNX2, which can be used to calculate a concentration of SP7, which can be used to calculate a concentration of ALP. It is further contemplated that additional osteoactivity marker concentrations may be calculated in other orders.

Various embodiments may be employed to offer services including providing reports that include information regarding osteoactivity of a sample. For example, a sample of biological material may be received, initial concentrations of various osteoactivity markers may be measured, second concentrations of one or more osteoactivity markers may be calculated in accordance with one or more embodiments described herein, and an output may be reported. The information regarding the osteoactivity of the sample may be, for example, predicted concentrations of one or more osteoactivity markers at a given point in time, the rate of change for one or more osteoactivity marker, or other information related to the osteoactivity of the sample.

### EXAMPLES

In order that various embodiments may be more readily understood, reference is made to the following examples which are intended to illustrate various embodiments, but not limit the scope thereof.

### Example 1

Adequate amounts of adipose-derived stem cells (ASCs) were removed from the cryotank for various examples. The tubes were placed immediately in a 37° C. water bath for 90 seconds with gentle agitation to thaw. Contents of all tubes were combined with 5 mL of prewarmed media and placed in 15 mL conical tubes. The cell/media suspension was centrifuged at 2,700 rpm for 5 minutes at room temperature. The supernatant was aspirated from each tube being careful not to disturb each cell pellet. Cells were resuspended in 1 mL of media per 1.0×10⁶ cells. Next, 10 μL of resuspended cells were removed for counting using a hemocytometer and the number of cells was recorded.

Additionally, 25 μL of the resuspended cells in media was saved into cryovials for use Western blot and microplate reader analyses. The cryovials were filled to 1.8 mL with media, and cells were counted using Scepter before being stored at −4° C. for analysis within two weeks, or −40° C. for analysis within six months.

Cells were resuspended, counted, and plated into pre-warmed T75 flasks containing 8 mL of media at a seeding density of approximately 500,000 cells/flask. Flasks were incubated for 4 hours before an initial check for confluency was conducted. Cells were checked daily for confluency, viability, and attachment using an EVOS microscope. Flasks were kept in the incubator, and media was replaced every business day with 18 mL on weekdays and 20 mL for weekends.

Cells were passaged to new flasks every 72-96 hours, or when the cells reached 90-100% confluency. For passage of cells to new flasks, the media was aspirated and saved at −4° C. for analysis. Room temperature trypsin (7.5 mL) was added to each flask and placed in the incubator for 6 minutes. The flasks were bumped to assist with the lifting of cells. Cell lifting was confirmed using the EVOS microscope. If cells had not lifted, they were incubated for 30 seconds more and reassessed.

Once the cells lifted, the trypsin was neutralized using 7.5 mL of media, and the contents were swirled into a corner of the flask for aspiration to a conical tube. Cells were then centrifuged at 2,700 rpm for 5 minutes and resuspended as detailed hereinabove. Feeding and passaging was repeated for as many passages as the cells allowed.

An adequate amount of fresh media was placed in each tube and the cells were resuspended within the media. The cells were then counted using a disposable hemocytometer to get an estimate of cell density per milliliter of media.

A Simple Western is an automated Western—no gels, no transfer devices, no blots, no film and no manual analysis. Samples were loaded in the automated system and start was pressed. The automated system automated all steps of the process including sample loading, size-based protein separation, immunoprobing, washing, detection and data analysis for up to 12 or 25 samples simultaneously based on equipment capabilities. Simple Western assays took place in a capillary. Samples were treated with sodium dodecyl sulfate (SDS) and dithiothreitol (DTT) and then heat denatured. Assay reagents and prepared samples were loaded into an assay plate and placed in the automated system. The automated system loaded samples into the capillary automatically. Proteins were separated by size as they migrated through both a stacking and separation matrix. The separated proteins were then immobilized to the capillary wall via a proprietary, UV capture method. Target osteoactivity markers were identified using a primary antibody and immunoprobed using an HRP-conjugated secondary antibody and chemiluminescent substrate. The resulting chemiluminescent signal was detected and quantitated. Analysis was automatically performed and results were presented in a corresponding software package.

Raw signals pertaining to marker concentration were normalized by cell population and/or particle density. This resulted in a corrected signal pertaining to marker concentration, which was used to derive the equations described above and below.

### Example 2

The equations derived in Example 1 were then tested for compatibility with other cell types. Human fibroblasts (FBs), bone marrow-derived stem cells (BMSCs), and adipose-derived stem cells (ASCs) were cultured as described hereinabove. Cell population and viability were measured using an automated cell counter. Total protein content was measured using a microplate reader. Osteoactivity markers BMP2, BMP7, BMP8a, pSMAD, DLX5, RUNX2, SP7, and ALP were measured by Western Blot. Raw signals pertaining to marker concentration were normalized by cell population and/or particle density.

For each of the osteoactivity markers, initial concentrations were measured. In addition, the additional concentration measurements for the osteoactivity markers were taken over time. A rate of change for each osteoactivity marker was calculated and plotted for the experimental data.

Initial concentrations for each of the osteoactivity markers were also input into the equations described hereinabove and the constants in each of the equations were optimized using GRG algorithms. The theoretical rate of change obtained from the equation was plotted along with the experimental data for each osteoactivity marker.

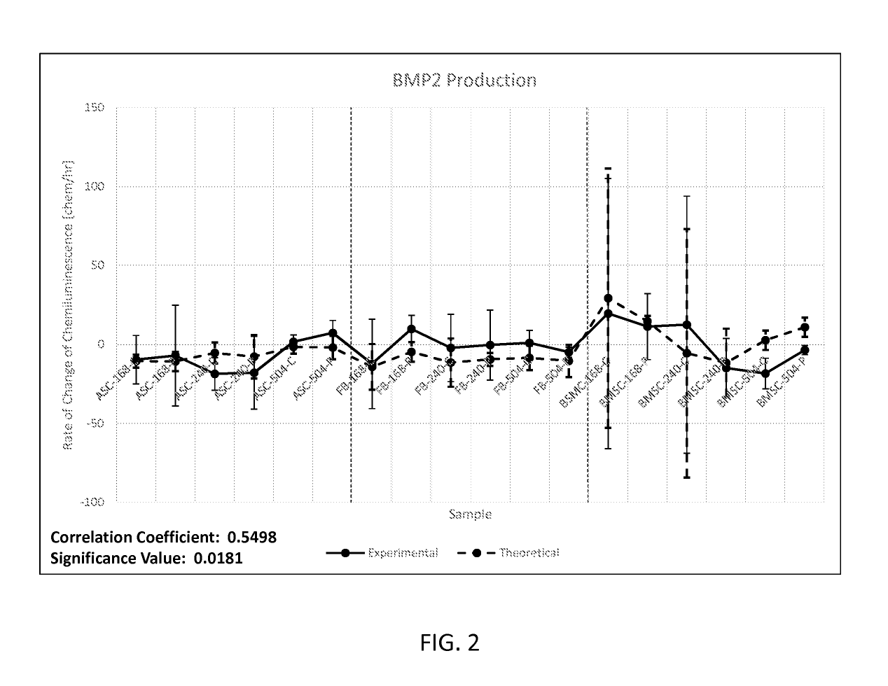

FIG. 2 illustrates a plot showing the rate of change of BMP2 for 18 samples encompassing three cell types. For the theoretical rate of change, v₁₀ was 2.21014×10⁻⁵, v₁₁ was 0.039717943, and v₁₂ was 36.11683. K₁₀ was 0.01461, K₁₁ was 6.312×10⁻⁵, and K₁₂ was 0.01659. Additionally, r₁₀ was 0.006352, r₁₁ was 0.009102, r₁₂ was 0.256916, and d₅ was 0.003172. As shown in FIG. 2, these values resulted in a correlation coefficient of 0.5498 and a significance value of 0.0181.

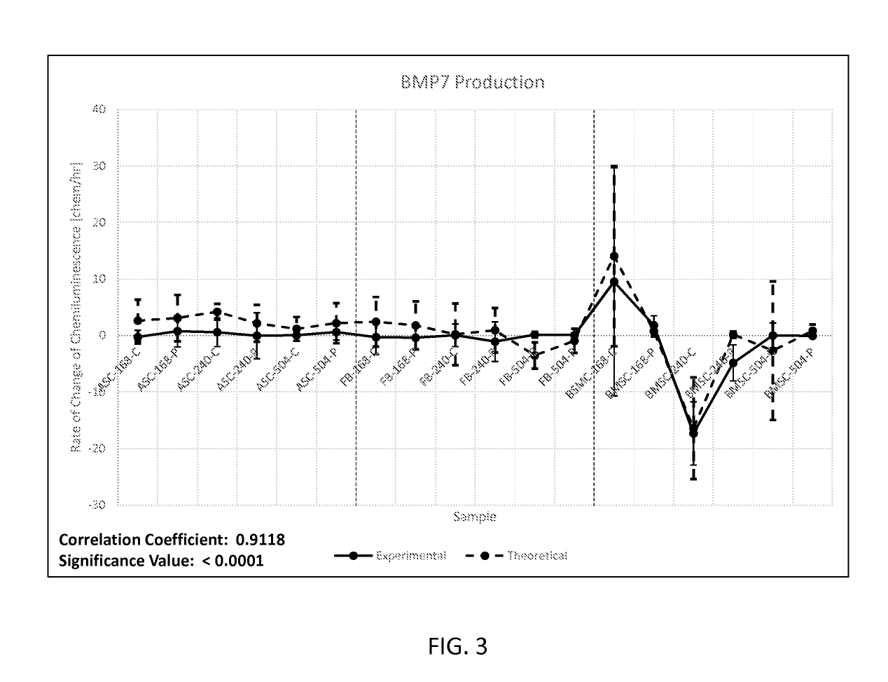

FIG. 3 illustrates a plot showing the rate of change of BMP7 for 18 samples encompassing three cell types. For the theoretical rate of change, v₁₃ was 8.23754×10⁻⁵, v₁₄ was 4.433923, and v₁₅ was 85.46909. K₁₃ was 0.11476, K₁₄ was 2.539×10⁻⁵, and K₁₅ was 9.014×10⁻⁵. Additionally, r₁₃ was 0.006352, r₁₄ was 0.069699, r₁₅ was 0.298225, and d₆ was 0.01106. As shown in FIG. 3, these values resulted in a correlation coefficient of 0.9118 and a significance value of less than 0.0001.

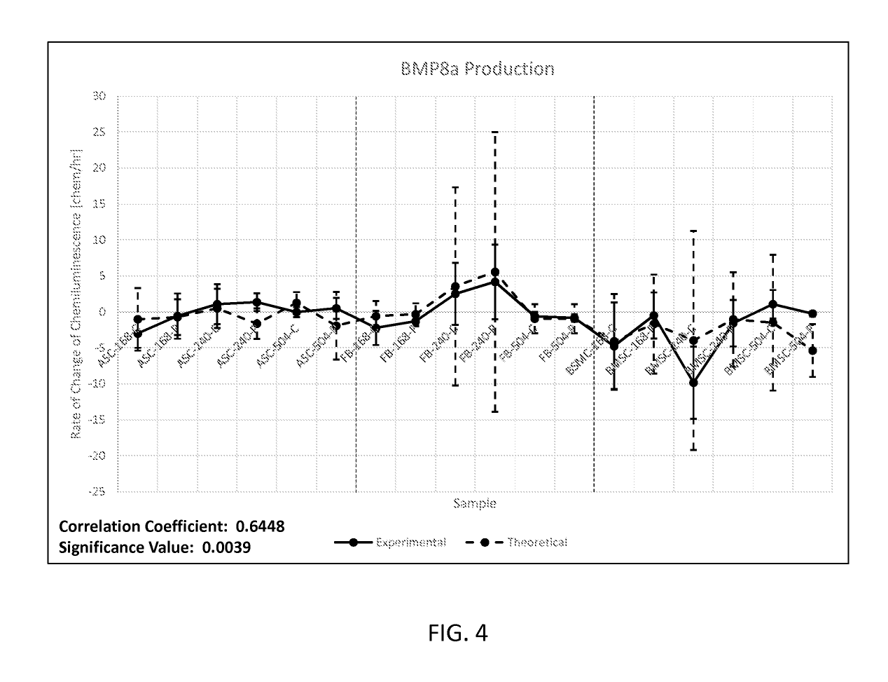

FIG. 4 illustrates a plot showing the rate of change of BMP8a for 18 samples encompassing three cell types. For the theoretical rate of change, v₁₆ was 48.62987, v₁₇ was 0.0, and v₁₈ was 2.53709×10⁻¹¹. K₁₆ was 0.281152, K₁₇ was 6.221×10⁻⁵, and K₁₈ was 2.21×10⁻⁴. Additionally, r₁₆ was 0.953747, r₁₇ was 0.0, r₁₈ was 0.001042, and d₇ was 0.043483. As shown in FIG. 4, these values resulted in a correlation coefficient of 0.6448 and a significance value of 0.0039.

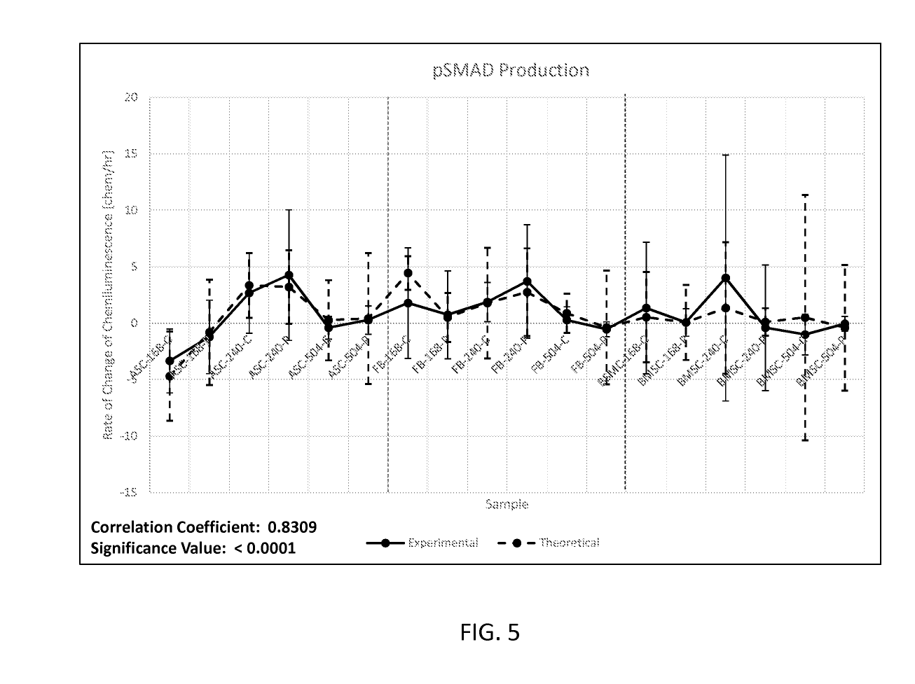

FIG. 5 illustrates a plot showing the rate of change of pSMAD for 18 samples encompassing three cell types. For the theoretical rate of change, v₁ was 5.127843, v₂ was 1.32456, and v₃ was 1.044437. K₁ was 0.076893, K₂ was 0.030286, and K₃ was 0.001272. Additionally, d₁ was 0.014868. As shown in FIG. 5, these values resulted in a correlation coefficient of 0.8309 and a significance value of less than 0.0001.

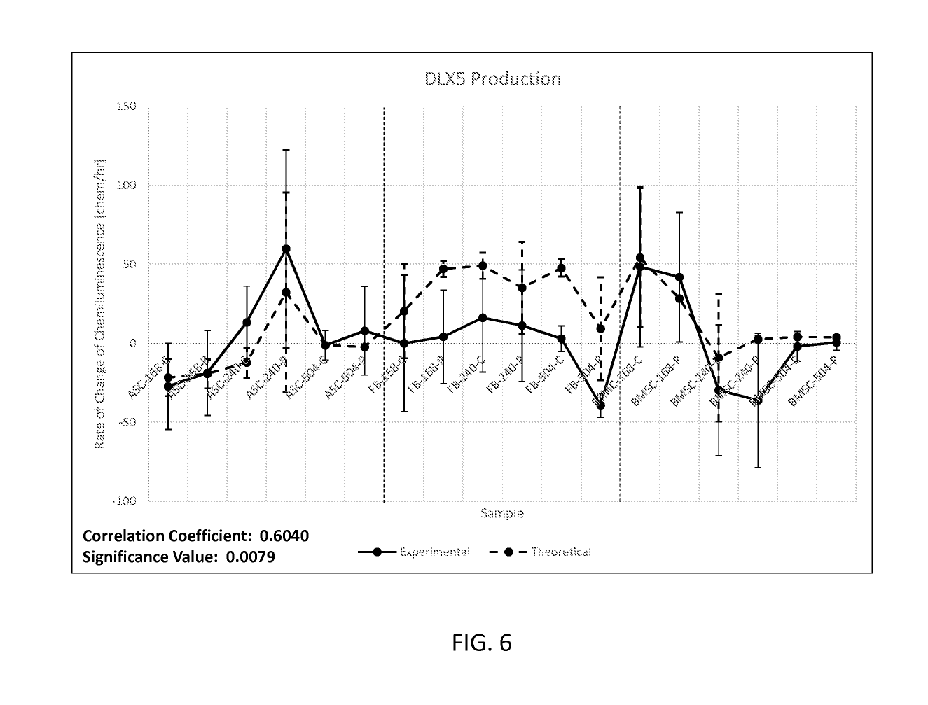

FIG. 6 illustrates a plot showing the rate of change of DLX for 18 samples encompassing three cell types. For the theoretical rate of change, v₆ was 21.75414, and K₆ was 20.72771. Additionally, d₃ was 0.00219. As shown in FIG. 6, these values resulted in a correlation coefficient of 0.6040 and a significance value of 0.0079.

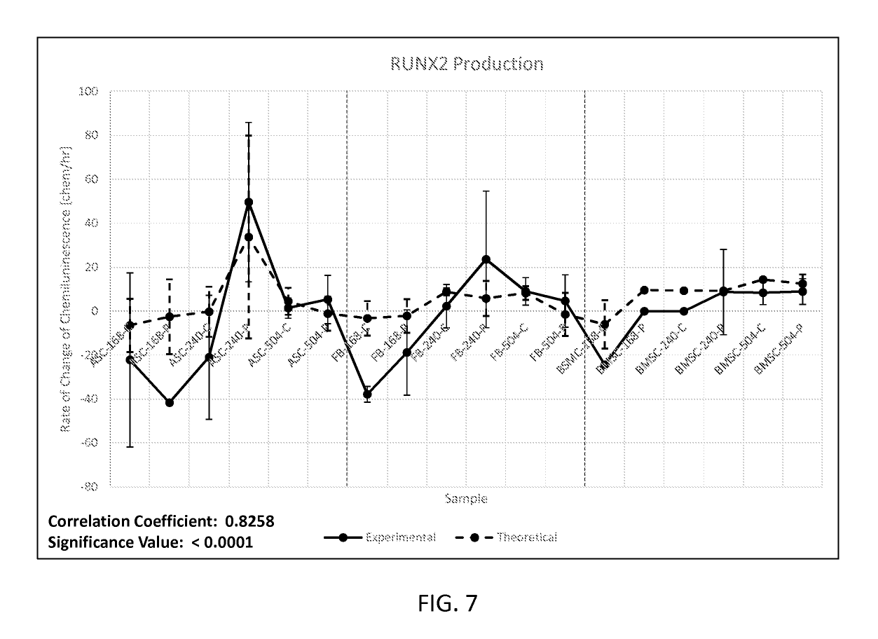

FIG. 7 illustrates a plot showing the rate of change of RUNX2 for 18 samples encompassing three cell types. For the theoretical rate of change, v₄ was 5.210283 and v₅ was 9.169164. K₄ was 5589.265 and K₅ was 0.30042. Additionally, d₂ was 0.003496. As shown in FIG. 7, these values resulted in a correlation coefficient of 0.8258 and a significance value of less than 0.0001.

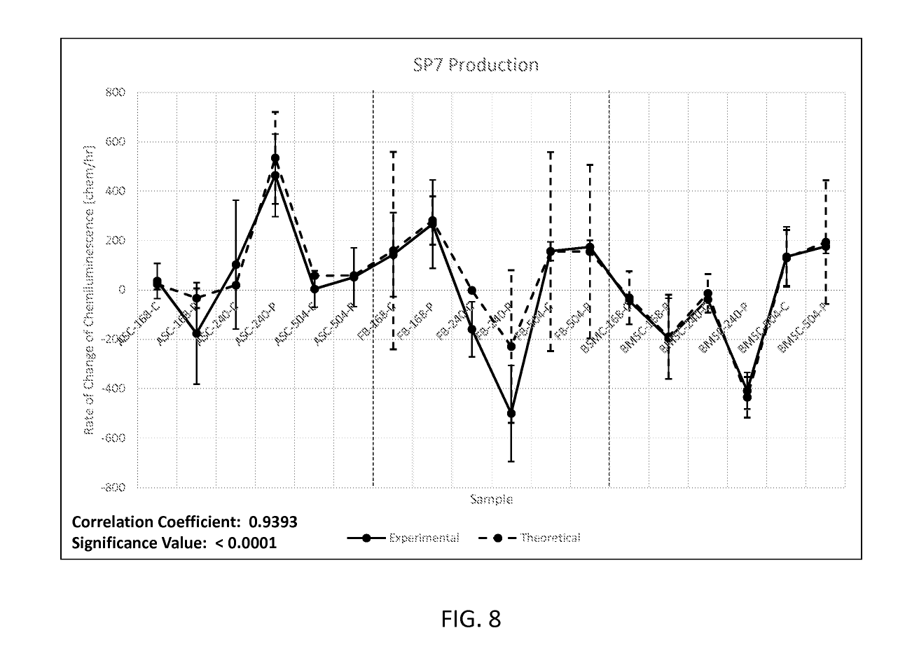

FIG. 8 illustrates a plot showing the rate of change of SP7 for 18 samples encompassing three cell types. For the theoretical rate of change, v₇ was 94.65879, v₈ was 0.040877, and v₉ was 16.86214. K₇ was 0.19388, K₈ was 2.483507, and K₉ was 2.483507. Additionally, d₄ was 0.008574. As shown in FIG. 8, these values resulted in a correlation coefficient of 0.9393 and a significance value of less than 0.0001.

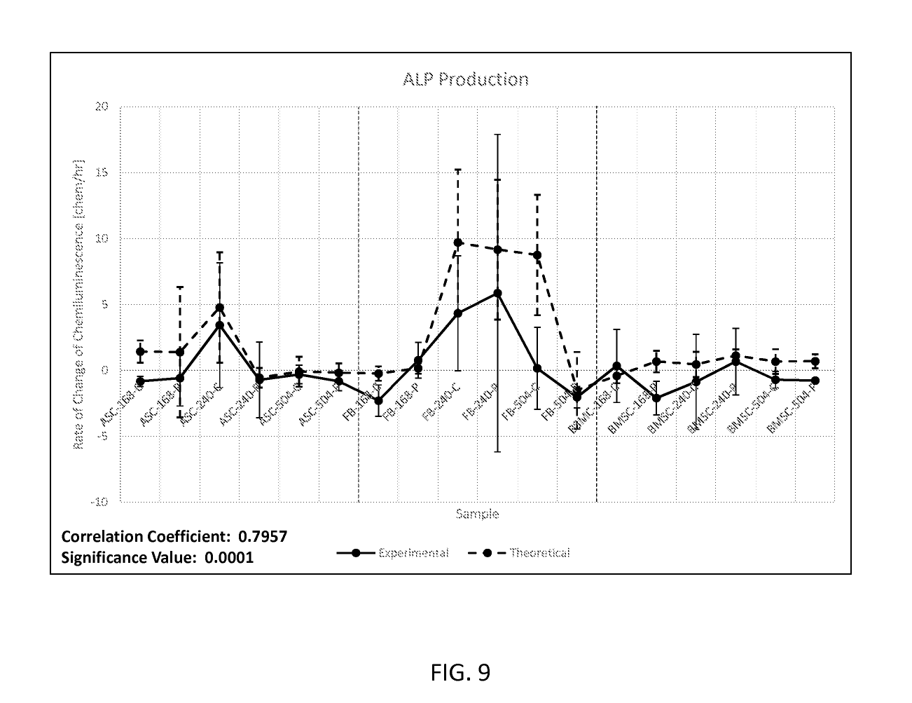

FIG. 9 illustrates a plot showing the rate of change of ALP for 18 samples encompassing three cell types. For the theoretical rate of change, v₂₀ was 0.133888 and v₂₁ was 0.174175. K₂₀ was 0.014929 and K₂₁ was 1.25973×10⁻⁸. Additionally, d₈ was 0.003346. As shown in FIG. 9, these values resulted in a correlation coefficient of 0.7957 and a significance value of 0.0001.

Accordingly, the equations set forth hereinabove can be used to reliably calculate the rate of change of various osteoactivity markers with a high degree of accuracy. In various embodiments, the rate of change can be determined with a significance value of less than 0.0001. Therefore, concentrations of these osteoactivity markers may be determined using calculations instead of via experimental data with a high degree of accuracy, thereby resulting in time and cost savings.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

1. A method of predicting an amount of a target osteoactivity marker, the method comprising:
measuring an initial concentration of the target osteoactivity marker at a time t₀;
measuring an initial concentration of at least one additional osteoactivity marker at the time t₀; and
calculating a second concentration of the target osteoactivity marker at a time t₁ based on the initial concentration of the target osteoactivity marker at the time t₀ and the initial concentration of the at least one additional osteoactivity marker at the time t₀.

2. The method of claim 1, wherein the target osteoactivity marker comprises DLX5, and wherein the at least one additional osteoactivity marker comprises pSMAD1/5.

3. The method of claim 1, wherein measuring the initial concentration of the at least one additional osteoactivity marker comprises measuring the initial concentration of at least two additional osteoactivity markers.

4. The method of claim 3, wherein the target osteoactivity marker comprises RUNX2, and wherein the at least two additional osteoactivity markers comprise pSMAD1/5 and DLX5.

5. The method of claim 3, wherein the target osteoactivity marker comprises ALP, and wherein the at least two additional osteoactivity markers comprise RUNX2 and SP7.

6. The method of claim 3, wherein the target osteoactivity marker comprises pSMAD1/5, and wherein the at least two additional osteoactivity markers comprise BMP2, BMP7, and BMP8a.

7. The method of claim 3, wherein the target osteoactivity marker comprises SP7, and wherein the at least two additional osteoactivity markers comprise pSMAD1/5, RUNX2, and DLX5.

8. The method of claim 3, wherein the target osteoactivity marker comprises BMP2, BMP7, or BMP8a, and wherein the at least two additional osteoactivity markers comprise RUNX2, DLX5, and SP7.

9. The method of claim 1, wherein the at least one additional osteoactivity marker comprises RUNX2.

10. The method of claim 1, wherein the at least one additional osteoactivity marker comprises DLX5.

11. A method of predicting an amount of a target osteoactivity marker, the method comprising:
receiving a sample comprising biological material;
measuring an initial concentration of the target osteoactivity marker in the sample at a time t₀;
measuring an initial concentration of at least one additional osteoactivity marker in the sample at the time t₀;
calculating a second concentration of the target osteoactivity marker at a time t₁ based on the initial concentration of the target osteoactivity marker at the time t₀ and the initial concentration of the at least one additional osteoactivity marker at the time t₀; and
reporting an output comprising information regarding osteoactivity of the sample.

12. The method of claim 11, wherein the biological material comprises cell suspensions, tissue extracts, blood, urine, media in which cells were grown, and/or cellular waste.

13. The method of claim 11, wherein said calculating is performed using a computing device.

14. The method of claim 11, wherein the at least one additional osteoactivity marker comprises RUNX2 or DLX5.

15. The method of claim 11, wherein the target osteoactivity marker comprises pSMAD1/5, DLX5, RUNX2, ALP, BMP2, BMP7, BMP8s, or SP7.

16. The method of claim 11, wherein measuring the initial concentration of the at least one additional osteoactivity marker comprises measuring the initial concentration of at least two additional osteoactivity markers.

17. The method of claim 16, wherein the osteoactivity marker comprises RUNX2, ALP, pSMAD1/5, SP7, BMP2, BMP7, or BMP8a.

18. The method of claim 11, wherein measuring the initial concentration of the at least one additional osteoactivity marker comprises measuring the initial concentration of at least three additional osteoactivity markers.

19. The method of claim 18, wherein the target osteoactivity marker comprises pSMAD1/5, SP7, BMP2, BMP7, or BMP8a.

20. The method of claim 11, further comprising:
calculating a second concentration of the at least one additional osteoactivity marker at a time t₁ based on the initial concentration of the target osteoactivity marker at the time t₀, the initial concentration of the at least one additional osteoactivity marker at the time t₀, and the second concentration of the target osteoactivity marker at a time t₁.
